(19) United States
(12) Patent Application Publication
(10) Pub. No.: US 2026/0151533 A1
(43) Pub. Date: Jun. 4, 2026

(54) SYNTHESIS AND CHARACTERIZATION OF GLYCOSAMINOGLYCAN MIMETIC VARIANTS TO PROMOTE CHONDROGENESIS

(71) Applicants: The Trustees of Columbia University in the City of New York, New York, NY (US); New Jersey Institute of Technology, Newark, NJ (US)

(72) Inventors: Treena Arinzeh, West Orange, NJ (US); Richard Vincent, Union, NJ (US); George Collins, Maplewood, NJ (US)

(21) Appl. No.: 19/405,685

(22) Filed: Dec. 2, 2025

Related U.S. Application Data
(60) Provisional application No. 63/726,855, filed on Dec. 2, 2024.

(51) Int. Cl. A61L 27/26; A61L 27/22; A61L 27/54; C08L 1/02; C08L 3/04; C08L 5/08; C08L 89/06

(52) U.S. Cl. CPC A61L 27/26; A61L 27/222; A61L 27/54; C08L 1/02; C08L 3/04; C08L 5/08; C08L 89/06; A61L 2300/414; A61L 2400/06; A61L 2430/06

(57) ABSTRACT

Compositions and methods for improving chondrogenesis, or treating osteoarthritis, or treating joint degradation or treating an osteochondral defect in a subject.

### CROSS-REFERENCE TO RELATED APPLICATIONS

This application claims benefit of U.S. Provisional Application No. 63/726,855, filed Dec. 2, 2024, the contents of which are hereby incorporated by reference.

### STATEMENT OF GOVERNMENT SUPPORT

This invention was made with government support under grant number AR077056 awarded by the National Institutes of Health and grant number 1548571 awarded by the National Science Foundation. The government has certain rights in the invention.

### BACKGROUND

Tissue engineering strategies to treat cartilage damage remain inadequate because of the difficulty in regenerating fully functional cartilage tissue. New approaches are needed.

### SUMMARY

A composition for chondrogenesis, comprising a sulfated chitin, sulfated starch, sulfated cellulose or sulfated dextran and, optionally, a carrier.

An injectable composition for chondrogenesis or articular cartilage repair or treating an osteochondral defect, the injectable fiber composition comprising sulfated chitin, sulfated starch, sulfated cellulose or sulfated dextran and a carrier. In some embodiments, the injectable composition is a scaffold. In some embodiments, the injectable composition comprises fibers. In some embodiments, the injectable composition comprises gelatin fibers.

A scaffold for chondrogenesis or articular repair comprising sulfated starch with a degree of sulfation of 1.5 per monomer of starch.

A method of improving chondrogenesis, or treating osteoarthritis, or treating joint degradation or treating an osteochondral defect in a subject in need thereof, the method comprising administering a composition comprising sulfated chitin, sulfated starch, sulfated cellulose or sulfated dextran described herein to a cartilage containing area, to a joint surface or to an osteochondral defect of the subject.

### BRIEF DESCRIPTION OF THE DRAWINGS

FIGS. 1A-1C: Structures A-C cellulose sulfate, starch sulfate and chitin sulfate, respectively. R=SO₃Na or H. Na is a counterion. Structures were made using Marvin Sketch [23].

FIGS. 2A-2C: FTIR spectra of starting materials and sulfated variants with varying degrees of sulfation (DOS). A) Cellulose and partially sulfated cellulose (pSC) materials, pSC DOS 0.5 and pSC DOS 1.1. B) Starch and partially sulfated starch materials, SS DOS 0.75, and SS DOS 1.5. C) Chitin and partially sulfated chitin (ChS) materials, ChS DOS 0.9 ad ChS DOS 0.975. All sulfated compounds show peak at 1220 cm⁻¹. Peak intensity increases with DOS.

FIG. 3: Raman spectra of the sulfated variants. All compounds show the presence of sulfate peaks at 1050 cm⁻¹. Spectra of pSCs and SS also show a peak around 800-850 cm⁻¹ suggesting the presence of sulfate groups on the 6th carbon (C6) of the monomer unit. SS shows a peak in the range of 920-960 cm⁻¹ associated with α-glycosidic bond vibrations.

FIGS. 4A-4C: Carbon NMR of the three sulfated compounds. A) pSC 0.5 and 1.1 carbon spectra show a broad peak at C6 sulfation at 66-70 ppM. C2 sulfation is also observable within a peak range of 75-83 ppM. B) SS 0.75 and 1.5 spectra depict C6 sulfation within the peak range of 68-72 ppM. Similarly, the spectra for starch-based variants reveal C2 sulfation within the peak range of 77-81 ppM. C) Spectra of the chitin-based variants reveal C6 sulfation within the peak range of 67-70 ppM and substitution of C2 at 77-82 ppM.

FIGS. 5A-5B: Cell number for compounds at A) low DOS (0.5, 0.75 and 0.9 DOS for pSC, SS and ChS, respectively) and B) high DOS (1.1, 1.5 and 0.975 DOS for pSC, SS, and ChS, respectively) (Values represent mean±standard deviation). *Significantly greater than GM at the time point (p<0.05)). #Significantly lower than GM at the time point (p<0.05).

FIGS. 6A-6B: A) Low DOS variant of pSC, SS, ChS. Live/dead images of cells cultured on tissue culture plate supplemented with increasing concentrations of pSC 0.5, SS 0.75 or ChS 0.9. Green=live cells, Red=dead cells. Scale bar=500 μm. All images taken at 4× magnification. B) High DOS variant of pSC, SS, ChS. Live dead images of cells cultured on tissue culture plate supplemented with increasing concentrations of pSC 1.1, SS 1.5 or ChS 0.975. Green=live cells, Red=dead cells. Scale bar=500 μm. All images taken at 4× magnification.”

FIG. 7: Histological staining of representative pellets after supplementation with 0.01% GAG mimetic at day 28. H&E, proteoglycan (Safranin O), Collagen Types I and II immunostaining. 20× Magnification.

FIGS. 8A-8D: A) Cell number of pellets supplemented with 0.01% (w/v) of GAG mimetic compared to CCM+ control at day 28. B) Total sGAG produced in cell pellets C) Total sGAG produced per cell. D) Heat map of collagen production (log₁₀, μg) of pellets supplemented with 0.01% GAG mimetic compared to CCM+ control. *Significantly higher than the control CCM+ (p<0.05), #Significantly lower than the control CCM+ (p<0.05) **Significantly higher than the control CCM+ (p<. 005), A significantly higher than all other groups (p<0.05), B significantly higher than all groups excluding SS 1.5 (p<0.05).

FIGS. 9A-9C: MSC pellets cultured in CCM+ with high glucose (HG) or low glucose (LG) media containing GAG-mimetics—DS or SS. (A) intracellular ATP, B) Col II Production, and C) Col I Production. a. Statistically different over time (p<0.05). b. Statistically higher than respective LG group (p<0.05). c. Statistically higher than respective CCM+ groups (p<0.05).

FIG. 10: Histological staining of pellets after supplementation with 0.01% GAG variant and either high or low concentrations of glucose at day 28. H&E, Safranin O (SafO) immunostains. 10× Magnification. Scale bar 100 μm.

FIGS. 11A-11D: A) Cell count, B) overall sulfated GAG content, C) Col II production, and D) Col I production on fragmented fibers. a. significantly different compared to respective gelatin group (p<0.05). b. significantly different compared to other fiber densities (p<0.05).

FIG. 12: H&E staining for chondrocyte conditioned-media and dextran sulfate.

FIG. 13: H&E staining for chondrocyte conditioned-media and dextran sulfate.

FIG. 14: SafO staining for chondrocyte conditioned-media and dextran sulfate.

FIG. 15: SafO staining for chondrocyte conditioned-media and dextran sulfate.

FIG. 16: Col II staining for chondrocyte conditioned-media and dextran sulfate.

FIG. 17: Col II staining for chondrocyte conditioned-media and dextran sulfate.

FIG. 18: Col I staining for chondrocyte conditioned-media and dextran sulfate.

FIG. 19: Col I staining for chondrocyte conditioned-media and dextran sulfate.

### DETAILED DESCRIPTION

Tissue engineering strategies to treat cartilage damage remain inadequate because of the difficulty in regenerating fully functional cartilage tissue. Sulfated glycosaminoglycans (GAGs), which are found in the native extracellular matrix, are known to interact with growth factors and thus, promote chondrocyte function. Native GAGs have been explored as viable scaffold materials for tissue repair applications. However, it is not known which structural feature(s) in GAGs are critical for promoting chondrogenesis. Therefore, this study generated GAG mimetics that vary in glycosidic linkage geometry and monomer ring substitution and were evaluated for their effect on mesenchymal stem cell (MSC) chondrogenesis for their potential use in cartilage tissue engineering applications. GAG mimetics were synthesized from cellulose (PSC), starch (SS), and chitin (ChS). pSC has beta glycosidic linkages, SS has alpha glycosidic linkages and ChS has beta-glycosidic linkages and N-acetyl substituted glucose monomers. Evaluated in soluble form in MSC pellet cultures, pSC and SS enhanced MSC chondrogenic differentiation as measured by the deposition of chondrogenic matrix components, collagen type II and GAG normalized to cell number, over ChS and the control culture media (without GAG mimetics). The higher degree of sulfation (DOS) in both the pSC and SS also had an effect on relative collagen type II deposition and GAG production. These data indicate beta and alpha glycosidic linkages are favorable for promoting chondrogenesis. Disclosed herein are GAG mimetics, including semi-synthetic, for chondrogenic differentiation where structural features should be considered for cartilage repair applications.

A composition for chondrogenesis, or for repair of chondral or osteochondral defects, comprising a sulfated chitin, sulfated starch, sulfated cellulose or sulfated dextran and, optionally, a carrier.

In some embodiments, the composition comprises a sulfated starch. In some embodiments, the composition comprises a sulfated cellulose. In some embodiments, the composition comprises a sulfated dextran. In some embodiments, the sulfated dextran is a 10 KDa dextran. In some embodiments, the composition sulfated chitin.

In some embodiments, comprising the sulfated starch, a degree of sulfation of the sulfated starch is 0.75 to 1.5 per monomer of starch. In some embodiments, the degree of sulfation of the starch is 1.5 per monomer of starch. Some embodiments comprise C6 sulfation of the starch.

In some embodiments, comprising the sulfated cellulose, a degree of sulfation of the cellulose is 0.5 to 1.1 per monomer of cellulose. Some embodiments comprise C6 and C2 sulfation of the cellulose.

In some embodiments, comprising the sulfated chitin, a degree of sulfation of the chitin is 0.9 to 0.975 per monomer of chitin. Some embodiments comprise C6 and C2 sulfation of the chitin.

In some embodiments, the composition is in the form of an injectable fiber or in the form of a scaffold. In some embodiments, the composition is in the form of a hydrogel.

An injectable composition for chondrogenesis or articular cartilage repair or treating an osteochondral defect, the injectable fiber composition comprising sulfated chitin, sulfated starch, sulfated cellulose or sulfated dextran and a carrier. In some embodiments, the injectable composition is a scaffold. In some embodiments, the injectable composition comprises fibers. In some embodiments, the injectable composition comprises gelatin fibers. Any source of gelatin is contemplated within the present embodiment including but not limited to porcine, porcine skin type A, bone, bovine hide, and bovine skin. Gelatin may be also obtained from tissue from the subject that the composition is injected into.

A scaffold for chondrogenesis or articular repair comprising sulfated starch with a degree of sulfation of 1.5 per monomer of starch.

In some embodiments, the scaffold comprises fibers. In some embodiments, the fibers are composed of gelatin. In some embodiments the fibers are electrospun and/or crosslinked. Chemical crosslinkers that are useful in this invention include those known in the art to be useful such as isosorbide-based epoxy crosslinker, genipin, 1-ethyl-3-(3-dimethylaminopropyl) carbodiimide (EDC), and glutaraldehyde. In some embodiments the fibers are present at 3 to 4 mg/ml of composition. In some embodiments the fibers are present at 3.4 to 3.6 mg/ml of composition. In some embodiments the fibers are present at 3.5 mg/ml of composition. In some embodiments the fibers are present at 0.5 to 10 mg/ml of composition. In some embodiments the fibers are present at 2.0 to 5 mg/ml of composition.

In some embodiments, the scaffold comprises one or more gelatin layers. In some embodiments, the gelatin layers are about 0.75 mm to 1.25 mm thick. In some embodiments, the gelatin layers are about 1 mm thick. In some embodiments, the gelatin layers are about 0.2 mm to 0.75 mm thick. The gelatin layers can be cut to the size of the defect if desired.

In some embodiments, the injectable fiber composition comprising fibers further comprises an amount of TGF-beta3.

In some embodiments, the scaffold for chondrogenesis further comprises an amount of TGF-beta3.

In some embodiments, the TGF-beta3 is recombinant and/or has the same sequence as a human TGF-beta3.

A method of improving chondrogenesis, or treating osteoarthritis, or treating joint degradation or treating an osteochondral defect in a subject in need thereof, the method comprising administering a composition comprising sulfated chitin, sulfated starch, sulfated cellulose or sulfated dextran described herein to a cartilage containing area, to a joint surface or to an osteochondral defect of the subject.

In some embodiments, the subject has osteoarthritis or has an inflammatory arthropathy or has relapsing polychondritis. In some embodiments, the method treats a knee joint, a hip joint or an elbow joint.

In some embodiments, the composition comprising sulfated chitin, sulfated starch, sulfated cellulose or sulfated dextran is administered into the cartilage containing area, joint surface or osteochondral defect.

In some embodiments, the composition comprising sulfated chitin, sulfated starch, sulfated cellulose or sulfated dextran is administered by an intra-articular injection.

In some embodiments, the composition comprising sulfated chitin, sulfated starch, sulfated cellulose or sulfated dextran is administered by surgery.

In some embodiments, the composition is in the form of one or more gelatin-scaffold layers comprising the sulfated chitin, sulfated starch, sulfated cellulose or sulfated dextran.

In some embodiments, the method effects an increase in chondrogenic matrix deposition.

In some embodiments, the subject has or is diagnosed with osteoarthritis.

In some embodiments of the methods, the subject is a human. In some embodiments, the subject is a horse, dog or cat.

In some embodiments, the scaffold described herein is cut or shaped to the shape and size of the chondral or osteochondral defect.

In some embodiments, the composition or scaffold further comprises cells (in non-limiting examples, chondrocytes, fibroblasts, progenitor cells, and/or stem cells) and/or one or more growth factors, cytokines, chemokines, antibiotics, DNA, or plasmids that induce directed growth and/or differentiation of cells, or vectors capable of delivering bioactive therapeutic genes to the product. In some embodiments, the composition also comprises collagen, hyaluronic acid, alginate, agarose, chitosan, gelatin, laminin, fibronectin or fibrin. In some embodiments, the composition does not contain collagen, hyaluronic acid, alginate, agarose, chitosan, gelatin, laminin, fibronectin or fibrin.

### Definitions

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about.” It is to be understood that such range format is used for convenience and brevity and should be understood flexibly to include numerical values explicitly specified as limits of a range, but also to include all individual numerical values or sub-ranges encompassed within that range as if each numerical value and sub-range is explicitly specified. For example, a ratio in the range of about 1 to about 200 should be understood to include the explicitly recited limits of about 1 and about 200, but also to include individual ratios such as about 2, about 3, and about 4, and sub-ranges such as about 10 to about 50, about 20 to about 100, and so forth. It also is to be understood, although not always explicitly stated, that the reagents described herein are merely exemplary and that equivalents of such are known in the art.

It must be noted that as used herein and in the appended claims, the singular forms “a,” “an,” and “the” include plural referents unless the context clearly dictates otherwise. Thus, for example, reference to “a light-harvesting molecule” includes a plurality of light-harvesting molecules.

Ranges recited herein are intended as continuous ranges, including every value between the minimum and maximum values recited, as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein, and in all instances such ratios, ranges, and ranges of ratios represent various embodiments of the present technology.

The term “about,” as used herein when referring to a measurable value such as an amount or concentration and the like, is meant to encompass variations of 20%, 10%, 5%, 1%, 0.5%, or even 0.1% of the specified amount.

Also as used herein, “and/or” refers to and encompasses any and all possible combinations of one or more of the associated listed items, as well as the lack of combinations when interpreted in the alternative (“or”).

As used herein, the terms “molecule”, “complex”, “compound”, and “product” are used interchangeably, and are intended to refer to a chemical entity, whether in the solid, liquid, or gaseous phase, and whether in a crude mixture or purified and isolated.

As used herein, “salt” refers to derivatives of the disclosed compounds or chemical structures wherein the parent complex is modified by making acid or base salts thereof.

As used herein, the term “solvate” refers to a physical association of a compound of this disclosure with one or more solvent molecules, whether organic or inorganic.

Compounds or chemical structures of the present technology, free form and salts thereof, may exist in multiple tautomeric forms, in which hydrogen atoms are transposed to other parts of the molecules and the chemical bonds between the atoms of the molecules are consequently rearranged. It should be understood that all tautomeric forms, insofar as they may exist, are included within the disclosure.

Throughout the specification and the appended claims, a given chemical formula or structure shall encompass all stereo and optical isomers and racemates thereof where such isomers exist. Unless otherwise indicated, all chiral (enantiomeric and diastereomeric) and racemic forms are within the scope of the disclosure. Many geometric isomers of C═N double bonds, ring systems, and the like can also be present in the complexes, and all such stable isomers are contemplated in the present disclosure.

As used herein, unless specified otherwise, the term “alkyl” means a branched or unbranched, saturated or unsaturated, monovalent or multivalent hydrocarbon group, including saturated alkyl groups, alkenyl groups and alkynyl groups. Examples of alkyl include, but are not limited to, methyl, ethyl, propyl, isopropyl, butyl, isobutyl, t-butyl, pentyl, hexyl, heptyl, octyl, nonyl, decyl, undecyl, dodecyl, ethenyl, propenyl, butenyl, isobutenyl, pentenyl, hexenyl, heptenyl, octenyl, nonenyl, decenyl, undecenyl, dodecenyl, ethynyl, propynyl, butynyl, isobutynyl, pentynyl, hexynyl, heptynyl, octynyl, nonynyl, decynyl, undecynyl, dodecynyl, methylene, ethylene, propylene, isopropylene, butylene, isobutylene, t-butylene, pentylene, hexylene, heptylene, octylene, nonylene, decylene, undecylene and dodecylene. In certain embodiments, the hydrocarbon group contains 1 to 30 carbons. In certain embodiments, the hydrocarbon group contains 1 to 20 carbons. In certain embodiments, the hydrocarbon group contains 1 to 12 carbons. In certain embodiments, the hydrocarbon group contains 1 to 6 carbons.

As used herein, unless specified otherwise, the term “cycloalkyl” means an alkyl which contains at least one ring, whether aromatic, or nonaromatic. In certain embodiments, a cycloalkyl is a saturated cycloalkyl group. In certain embodiments, a cycloalkyl group comprises 1 or more unsaturated bonds. In certain embodiments, a cycloalkyl group comprises an aryl group. Examples of cycloalkyl groups include, but are not limited to, benzene, cyclopropyl, cyclobutyl, cyclopentyl, cyclohexyl, cycloheptyl, cyclooctyl, cyclononyl, cyclodecyl, cycloundecyl and cyclododecyl. In certain embodiments, the hydrocarbon chain contains 1 to 30 carbons. In certain embodiments, the hydrocarbon group contains 1 to 20 carbons. In certain embodiments, the hydrocarbon group contains 1 to 12 carbons. In certain embodiments, the hydrocarbon group contains 1 to 6 carbons.

As used herein, unless specified otherwise, the term “heterocycloalkyl” means a cycloalkyl wherein at least one ring atom is a non-carbon atom. Examples of the non-carbon ring atom include, but are not limited to, S, O and N. Examples of heterocycloalkyl groups include, but are not limited to, 1,2-oxathiolane, 1,3-oxathiolane, azete, azetidine, aziridine, azirine, diazete, diazetidine, diazinane, diazine, diaziridine, diazirine, dioxane, dioxazole, dioxetane, dioxete, dioxine, dioxirane, dioxolane, dithiane, dithiazole, dithietane, dithiete, dithiin, dithiolane, dithiole, furan, furazan, hexazine, imidazole (imidazoline), imidazolidine, isothiazole, isothiazolidine, isoxathiole, isoxazole, morpholine, oxadiazole, oxane, oxatetrazole, oxathiane, oxathiin, oxathiole (oxathioline), oxazine, oxaziridine, oxazirine//oxirane, oxazole (oxazoline), oxazolidine isoxazolidine, oxetane, oxete, oxirene, pentazole, piperidine, pyran, pyrazole (pyrazoline), pyrazolidine, pyridine, pyridinium cation, pyrrole, pyrrolidine, pyrylium cation, tetrahydrofuran, tetrahydrothiophene, tetrahydrothiophene, tetrazine, tetrazole, thiadiazole, thiane, thiatetrazole, thiazine, thiazole (thiazoline), thiazolidine, thietane, thiete, thiirane, thiirene, thiomorpholine, thiophene, thiopyran, thiopyrylium cation, triazinane, triazine, triazoles, trioxane, trithiane.

As used herein, unless specified otherwise, the term “alkoxyl” means an alkyl, cycloalkyl or heterocycloalkyl, which contains one or more oxygen atoms. Examples of alkoxyl include, but are not limited to, —CH2-OH, —OCH3, —O-alkyl, -alkyl-OH, -alkyl-O-alkyl-, wherein the two alkyls can be the same or different.

As used herein, unless specified otherwise, the term “alkylthio” means an alkyl, cycloalkyl or heterocycloalkyl, which contains one or more sulfur atoms. Examples of alkylthio include, but are not limited to, —CH2-SH, —SCH3, —S-alkyl, -alkyl-SH, -alkyl-S-alkyl-, wherein the two alkyls can be the same or different.

As used herein, unless specified otherwise, the term “alkylcarbonyl” means an alkyl, cycloalkyl or heterocycloalkyl, which contains one or more carbonyl groups. Examples of alkylcarbonyl group include, but are not limited to, aldehyde group (—R′—C(O)—H), ketone group (—R′—C(O)—R″), carboxylic acid group (R′—COOH), ester group (—R″—COO—R′), carboxamide, (—R″—COO—N(R′)R″), enone group (—R″—C(O)—C(R′)═C(R″)R′″), acyl halide group (—R′—C(O)—X) and acid anhydride group (—R″—C(O)—O—C(O)—R′), wherein R′, R″, R′ and R″″ are the same or different alkyl, cycloalkyl, or heterocycloalkyl.

As used herein, unless specified otherwise, the term “alkylcarboxylate” means an alkyl, cycloalkyl or heterocycloalkyl, which contains one or more deprotonated carboxylic acid groups. Examples of alkylcarboxylate groups include, but are not limited to, carboxylate group (R′—COO—) wherein R′ is an alkyl, cycloalkyl, or heterocycloalkyl.

As used herein, unless specified otherwise, the term “aryl” means a chemical structure comprising one or more aromatic rings. In certain embodiments, the ring atoms are all carbon. In certain embodiments, one or more ring atoms are non-carbon, e.g. oxygen, nitrogen, or sulfur (“heteroaryl”). Examples of aryl include, without limitation, phenyl, benzyl, naphthalenyl, anthracenyl, pyridyl, quinoyl, isoquinoyl, pyrazinyl, quinoxalinyl, acridinyl, pyrimidinyl, quinazolinyl, pyridazinyl, cinnolinyl, imidazolyl, benzimidazolyl, purinyl, indolyl, furanyl, benzofuranyl, isobenzofuranyl, pyrrolyl, indolyl, isoindolyl, thiophenyl, benzothiophenyl, pyrazolyl, indazolyl, oxazolyl, benzoxazolyl, isoxazolyl, benzisoxazolyl, thiaxolyl, quanidino and benzothiazolyl.

As used herein, the term “substituted” refers to at least one hydrogen atom that is replaced with a non-hydrogen group, provided that normal valencies are maintained and that the substitution results in a stable compound. When a group is noted as “optionally substituted”, the group may or may not contain non-hydrogen substituents.

Tissue engineering has emerged as a promising strategy for the replacement of damaged or diseased tissue [1]. Glycosaminoglycans (GAGs) are polysaccharides that are present in the extracellular matrix (ECM) as components of proteoglycans (PG) and on the surface of cells [2, 3]. GAGs are integral regulators of many biochemical functions such as proliferation and differentiation. These biochemical processes are known to be dependent on GAG molecular structure. Tissue engineering strategies have used native GAGs as a biomaterial component for scaffolds [24]. Native GAGs can be isolated from animal sources but can have heterogeneity in GAG content and batch to batch variation [4-7]. In order to overcome these limitations, an alternative approach is the synthesis of GAG mimetics that incorporate structural features found in native GAGs where the degree of substitution can be controlled and can minimize batch to batch variation. In cartilage tissue engineering, one challenge is generating constructs that enhance chondrogenesis while minimizing hypertrophic differentiation [8]. It has been shown that constructs containing native GAGs [7, 9-11] or GAG mimetics [5, 12, 13] can enhance chondrogenesis in vitro and have shown promise in vivo, however it is not clear which structural features should be considered when synthesizing GAG mimetics for chondrogenic differentiation. In our previous studies, we have demonstrated that sulfated cellulose can promote chondrogenesis in vitro whether in soluble form [14] or when combined with biomaterials in the form of a scaffold [5, 15]. In addition, the higher molecular weight GAG mimetics derived from cellulose may be more favorable for chondrogenesis than lower molecular weight native GAGs due to enhanced sequestration of the growth factor TGF-β [16]. However, comparisons of GAG mimetics are needed that differ in structure that more closely represent native GAGs [13, 17, 18].

### Examples—I

This study investigated the synthesis and characterization of GAG mimetics derived from readily available polysaccharides that vary in structure. They were evaluated for supporting MSC chondrogenesis. Cellulose, chitin and starch are derived from cotton, shellfish, and potatoes, respectively. Upon sulfation, they incorporate structural features similar to those found in native GAGs, in particular chondroitin-6-sulfate (CSC) and heparin sulfate (HS) which are the predominant GAG components of the native cartilage ECM during chondrogenesis [19, 20]. CSC containing 6-O-sulfated N-acetyglucosamine (GlaNAc) and uronic acid (UA) disaccharide units bound by alternating β-1,4 or β-1,3 glycosidic linkages. HS is commonly composed of disaccharide repeats of the sulfated UA stereo-epimer iduronic acid (IA) and sulfated GluNAc disaccharide units bound by alternating β-1,4 and α-1,4-glycosidic linkages [21].

In this study, the structural features of interest are the stereochemistry of the glycosidic linkage and the monomer substitution. Cellulose is composed of β-D-glucose monomers linked by β-1,4 glycosidic bonds. Starch is also composed of the D-glucose monomers but contain predominantly α-1,4 glycosidic linkages. Chitin contains the amide derivative of the glucose monomer, N-acetylglucosamine (GluNAc), with β-1,4 glycosidic linkages. CSC and HS also have a degree of sulfation (DOS) of approximately 0.5 sulfates per monomer unit [22].

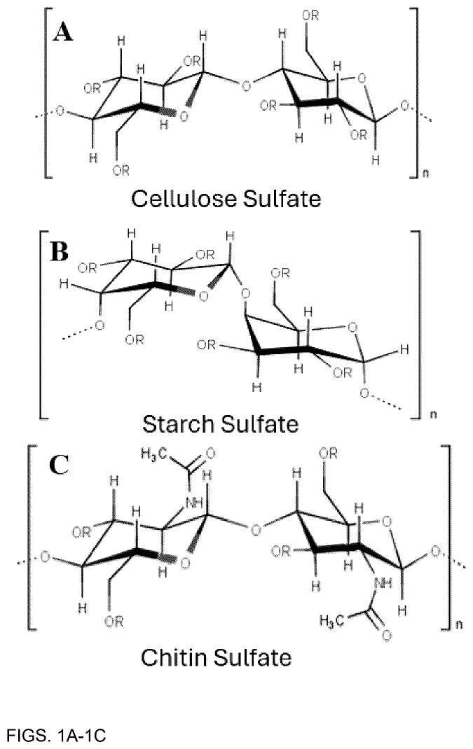

In this study, the GAG-mimetics had differences in stereochemistry and changes in their DOS (FIG. 1). The sulfated variants were evaluated for their effect on MSC chondrogenesis when used in soluble form in the media. Findings demonstrated that both alpha and beta glycosidic linkages with a high DOS were more favorable at promoting chondrogenesis at demonstrated by chondrogenic matrix deposition.

### Methods

### 2.1 Synthesis of Sulfated Cellulose, Starch, and Chitin

Sulfated cellulose was prepared similarly to previously reported protocols [15]. The sulfation of starch and chitin was performed using a modified version of previously reported methods [5, 24, 25]. Prior to sulfation, microcrystalline cellulose (Sigma-Adrich, St. Louis, MO, USA), starch (Sigma-Adrich, St. Louis, MO, USA) and chitin (Alpha Aesar, Ward Hill, MA, USA) were dried under vacuum at 120° C. for 24 hours.

2.1.1 Synthesis of Sulfated Cellulose: In brief, N, N-dimethylformamide (DMF) (Oakwood Chemical, West Columbia, SC, USA) was mixed with chlorosulfonic acid (HClSO₃) (Fisher, Waltham, MA, USA) under anhydrous conditions generating the sulfating agent. Cellulose was then dispersed in DMF (Oakwood) and stirred for 24 hours. The sulfating agent was then added at room temperature and allowed to react for 24 hours. The reaction was then quenched via the addition of supersaturated sodium acetate (Oakwood Chemical, West Columbia, SC, USA) in denatured ethanol (Fisher, Waltham, MA, USA) then vacuum filtered. The pulp was then dissolved in DI water and titrated to neutrality with 4M sodium hydroxide (Fisher). The resultant dispersion was purified by dialysis for 48 hours [5]. The stoichiometry range of the reaction was 0.77:1 mol agent/mol polymer (as calculated per monomer unit) to 1.22:1 mol agent/mol polymer (as calculated per monomer unit) to achieve polymer batches having approximately a DOS of 0.5 or 1.1, respectively or sulfates per monomer unit, respectively.

### 2.1.2 Synthesis of Sulfated Starch and Chitin

2.1.2.a. Sulfated Starch: Pyridine was mixed with HClSO₃ under anhydrous conditions, generating the sulfating agent pyridine sulfurtrioxide (PSO₃). The sulfating agent was then heated to 50-60° C. and then, starch was added to PSO₃ under anhydrous conditions. The stoichiometry range of the reactions were 1.22:1 mol agent/mol polymer (as calculated per monomer unit) to achieve a DOS of 0.75 and 2.54:1 to achieve DOS of 1.5, respectively. To form batches of starch sulfate (SS) at a DOS of 0.75, the reaction was undertaken for 24 hours prior to quenching and 96 hours for SS at DOS 1.5. After the formation of pyridinium salts, the reaction was quenched by the addition of DI water. The crude SS was then solubilized in DI water then precipitated with denatured ethanol (Fisher) and filtered. The resultant dispersion was purified by dialysis for 48 hours.

2.1.2.b. Sulfated Chitin: The sulfating agent, PSO₃, was prepared as described above. The sulfating agent was heated to 80-85° C. and chitin was added under anhydrous conditions. The stoichiometry of the reactions was 2:1 mol agent/mol polymer (as calculated per monomer unit) to achieve both 0.9 and 0.975 DOS, respectively. To form batches of chitin sulfate (ChS) at a DOS of 0.9, the reaction was undertaken for 1 hour prior to quenching and 2 hours for ChS at DOS 0.975. The reaction was then quenched by the addition of DI water, followed by titration to neutralize the reaction with 4N NaOH. The crude ChS was then solubilized with DI water then precipitated with denatured ethanol (Fisher) and filtered. The resultant dispersion was purified by dialysis for 48 hours.

### 2.2 Characterization of Sulfated Compounds

2.2.1 Elemental Analysis: Purified and filtered GAG mimetic compounds were analyzed by elemental analysis (Galbraith Laboratories, Inc., Knoxville, TN, USA) to determine overall % sulfur, carbon and oxygen. The DOS was determined using the empirical formula [5].

2.2.2 Gel Permeation Chromatography (GPC): Molecular weight were determined by size exclusion chromatography with refractive index detection using (SEC-IR, Polymer Solutions, Inc, Christianburg, VA, USA.), as previously described by our group [26].

2.2.3 FTIR and Raman Spectroscopy: All samples were analyzed as previously described [26]. To determine if there was sulfate group substitution in the crude materials after the sulfation reaction, all sulfated materials and non-sulfated starting materials were analyzed. FTIR was performed using Perkin Elmer FTIR-ATR 100 series (Perkin Elmer Life and Analytical science, Shelton, CT, USA). Data were collected from the range of 600-3200 cm⁻¹ with a spectral resolution of 5 cm⁻¹. The data were analyzed and plotted using Excel. Raman spectroscopy was run on all sulfated materials and non-sulfated starting materials as adapted from Mainreck et al [27]. Samples were analyzed using Thermo Fisher DXR Raman microscope (Thermo Electron Scientific Instruments LLC, Madison, WI. USA) with operating laser excitation of 780 nm at magnification of 100× through a long working distance objective. Data were collected from the range of 600 to 1700 cm⁻¹ with fifteen acquisitions of 30 s each. All chitin samples were photobleached for 2 hours prior to acquisition to minimize fluorescence.

2.2.4 Nuclear Magnetic Resonance Spectroscopy (¹³C-NMR and ¹H-NMR): Proton NMR (¹H-NMR) was used to determine the presence of impurities. Position of the sulfate group was analyzed by carbon-13 NMR (¹³C-NMR). The sulfated compound was placed into an NMR tube containing sodium trimethylsilylpropanesulfonate (DSS) as an internal NMR standard. 1H-NMR was analyzed using a Bruker Avance III HD 500 MHz NMR Spectrometer (Bruker, Billerica, MA, USA) equipped with 5 mm boron selective probe. Shims were adjusted according to Bruker procedures; 32 transients were recorded then Fourier transformed to yield proton spectra. Similarly, carbon nuclei were analyzed using z-restored spin-echo sequence on a 600 MHz NMR (Agilent, Santa Clara, CA, USA) equipped with an HCN cold probe after shims were adjusted according to Agilent's procedures. Afterwards, 12-18,000 transients were acquired and Fourier transformed to acquire spectra for all carbon nuclei [26]. All spectra were analyzed using Brucker Topspin software.

### 2.3 Cell Growth and Viability

Human MSC isolation and culture were conducted as previously described [5]. Briefly, human MSCs were obtained from human bone marrow aspirates (Lonza, Walkersville, MD, USA), male and female donors, aged 18-30 years. MSCs were expanded and cryopreserved until use. At passage 3, cryopreserved MSCs were thawed and expanded on tissue culture polystyrene flasks (NunC, Rochester, NY, USA) in general media (GM) comprising Dulbecco's modified Eagle's medium (DMEM); (Gibco, Carlsbad, CA, USA), 10% fetal bovine serum (Hyclone, Logan, UT, USA), and 1% antibiotic-antimycotic (Gibco) until 70-80% confluent. MSCs were trypsinized and then seeded onto 96 well tissue culture polystyrene plates (Fisher) at 2.11×10⁴ cells/cm². All sulfated compounds were resuspended in 1 L of DI water, sterilized with ethanol and dried using previously established methods [5, 26]. 0.1, 0.01 and 0.001 (w/v) % solutions of the sulfated compounds were then prepared in GM. Cultures were maintained for 14 days and the cell culture media was changed every 3 to 4 days. The control was GM without the GAG mimetic.

Cell number was determined at days 1, 7 and 14. Cell standards and samples were lysed with 0.1% Triton X-100 (Sigma-Adrich). The Quant-iT dsDNA Picogreen Assay Kit (Invitrogen, Carlsbad, CA, USA) was used to quantify cell number [5]. The picogreen reagent was added to the cell lysate according to the manufacturer's protocol. The fluorescence intensity of each sample was measured using a fluorescence microplate reader (FLX800, Biotek instruments, VT, USA) at 480 nm excitation and 520 nm emission. Fluoresence intensity was related to cell number using a standard curve.

Live/dead imaging was performed using the live/dead cytotoxicity and viability kit (Invitrogen), according to the manufacturer protocols. Briefly, ethidium-homodimer was diluted 1:500, and calcein 1:2000 in PBS. Solution was added to each sample and incubated at room temperature for 30 minutes. Cells were imaged using fluorescence microscopy (Nikon C1, Melville, NY, USA).

### 2.4 In Vitro Chondrogenesis

Cryopreserved MSCs were thawed and expanded to passage 3 on tissue culture polystyrene flasks in GM until 70-80% confluent. MSCs were trypsinized and suspended in Chondrogenic Complete Media (CCM−) composed of high-glucose DMEM (Invitrogen) supplemented with 1 mM sodium pyruvate (Sigma-Adrich, St. Louis, MO, USA), 0.35 mM L-proline (Sigma Aldrich), 4 mM L-glutamine (Invitrogen, NY, USA), 1% antibiotic-antimycotic (Invitrogen, NY, USA), 1% ITS-Premix consisting of insulin, human transferrin and selenous acid (Corning, MA, USA). MSCs were counted and seeded at 2×10⁵ cells in 500 μl of CCM− in a 15 mL polypropylene conical tube (USA Scientific, Ocala, Florida, USA). The tubes were then centrifuged to form pellets at 250×g, and the media was changed to (CCM+) which is CCM− with s 10 ng/ml of transforming growth factor beta (TGF-β3) (ProSpecbio, NJ, USA) with 0.01% wt./vol. GAG mimetics. The control condition was CCM+ without GAG mimetics. The media was changed every 3-4 days and the pellets were cultured until day 28.

2.4.1. Cell number and GAG production: Cell number and GAG production was determined at day 28. Papain solution containing 0.46% (wt/vol) Papain (Sigma-Adrich), 100 mM sodium phosphate dibasic (Sigma-Adrich), 10 mM Ethylenediaminetetraacetic acid disodium salt dihydrate (EDTA-2H₂O) (Sigma-Adrich) and 10 mM cysteine hydrochloride (Sigma-Adrich) was prepared for pellet digestion. To determine cell number, N=3 pellets were washed with PBS and then digested in 500 μl papain for 24 hours [15]. The Picogreen Assay Kit (Invitrogen) was used to quantify cell number of the digested pellets as described above [5]. The papain digested samples used for the Picogreen assay also were used to detect the sulfated GAG in each sample using the DMMB assay [28]. Chondroitin sulfate A (CSA) (Sigma-Adrich, St. Louis, MO, USA) was used as a standard and absorbances were measured at 595 nm using spectrophotometer (Emax, Molecular Devices, San Jose, CA).

2.4.2. Histology and immunohistochemical staining (IHC): For histology, pellets, n=2 per group, were fixed in 4% paraformaldehyde (Sigma-Adrich) overnight at 4° C. Pellets were processed for routine histology, embedded in paraffin, and sectioned. Sections were stained with haematoxylin and eosin (H&E), safranin O and toluidine blue. To observe collagen types I and II deposition, the samples were immunostained with Anti-Collagen I antibody (COL1) (Abcam, Waltham, MA, USA) or Anti-Collagen II antibody (Abcam, Waltham, MA, USA). Images were taken with an optical microscope at 10× magnification for pellets at 28 days.

2.4.3. Collagen Types I and II Quantification: Quantitative measurement of collagen type I and II of the pellets was performed by ELISA. Samples were evaluated for the production of collagen type I (COL1) and collagen type II (COL2) using sandwich enzyme-linked immunosorbent assay (ELISA) (Chondrex, Woodinville, WA, USA) after day 28 of culture. Samples were digested as described [5, 15]. Briefly, N=4 samples were pooled and placed into a solution of 0.1 mg/ml pepsin (Chondrex, Woodinville, WA, USA) in 0.05 mM Acetic acid (Sigma-Adrich, St. Louis, MO, USA) and allowed to digest for 96 hours. After pepsin digestion, the samples were also treated with elastase (Chondrex) for 24 hours, followed by treatment with buffered normal goat serum (NBS) (Chondrex1× Tris buffered saline (TBS) (Sigma-Adrich). ELISA kits for human collagen type I (Chondrex) and human collagen type II (Chondrex) were used. Samples were run in triplicate. Fluorescence intensity was measured using Emax (Molecular Devices) at 520 nm. Standard curve was used to determine the amount of protein.

### 2.5 Statistical Analysis

Statistical analysis was performed using two-way analysis of variance (ANOVA) to determine significant differences between groups and time (p<0.05). Shapiro Wilk test and Levene's equal variance test was used to determine the normality. Multiple comparisons between groups were made using Tukey's posthoc test (p<0.05). Statistical analysis was performed using GraphPad Prism (La Jolla, CA, USA) software. All raw data and plots were processed in Microsoft Excel or GraphPad Prism software. All values are reported as mean±standard deviation.

### Results

### 3.1 Synthesis and Characterization of Sulfated Glycosaminoglycan Mimetics

Sulfated cellulose, starch and chitin were synthesized. Elemental analysis is shown in Table 1. For cellulose sulfates (PSC), sulfur percentages of 6.72 and 9.03% were determined, which corresponded to a DOS of 0.5 and 1.1, respectively. Similarly, the starch sulfates (SS) had sulfur percentages of 8.74 and 11.74%, which corresponded to a DOS of 0.75 and 1.5, respectively. For the chitin sulfate (ChS) groups, the sulfur percentages were 6.44 and 6.51%, which corresponded to a DOS of 0.9 and 0.975, respectively.

TABLE 1
Elemental analysis, DOS, and molecular wt. of sulfated compounds

| Group | pSC | pSC | SS | SS | ChS | ChS |
|---|---|---|---|---|---|---|
| Elemental | 2.43 | 2.42 | 2.94 | 1.42 | 4.2 | 4.74 |
| Analysis C/S | | | | | | |
| DOS | 0.5 | 1.1 | 0.75 | 1.5 | 0.9 | .975 |
| Molecular Wt | 365 | 34.2 | 2.6 | 1.95 | 22 | 4.120 |
| (kDa) | | | | | | |

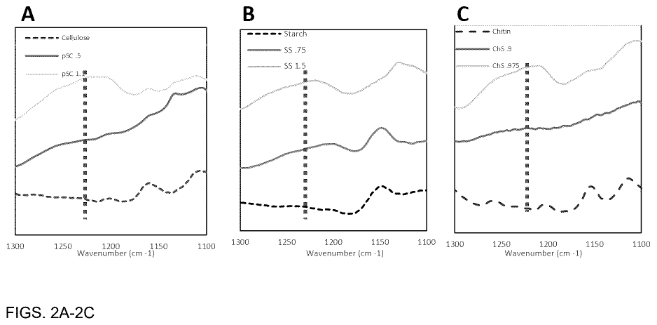

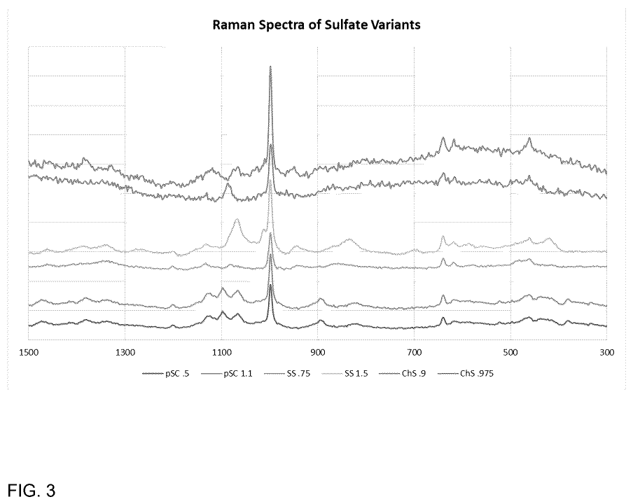

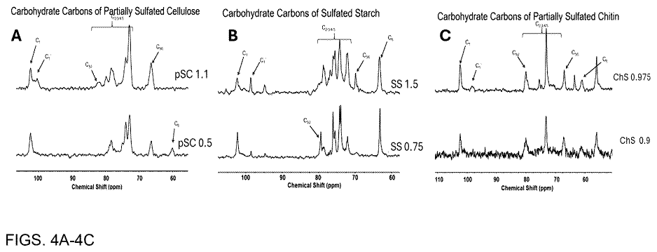

FTIR spectra of the synthesized variants showed the pronounced peaks associated with the sulfate substitutions in the range of 1220-1300 cm⁻¹ (FIG. 2). The peak intensity was reduced for the lower DOS sulfated variants. Raman spectroscopy was used to confirm the FTIR results. The Raman spectra of the sulfated variants showed a peak associated with sulfation (S═O stretching) at the Raman shift of 1050 cm⁻¹ (FIG. 3) [27]. For pSC and SS at both DOS, Raman spectra showed a small peak in the range of 800-850 cm⁻¹, which is consistent with sulfation at the 6th carbon position (C6) of the glucose monomer unit [26]. Based on published reports of chemical shifts of polysaccharides with alpha glycosidic linkages [27, 29, 30], the Raman spectra for SS shows a peak in the range of 920-960 cm⁻¹ associated with α-glycosidic bond vibrations. SS, pSC and ChS spectra all share a peak in the range of 470-490 cm⁻¹ which is associated with C1-O—C4 glycosidic linkage stretching reviewed in [30].

To analyze the presence of impurities in the sulfated products, proton NMR was implemented. All proton NMR revealed the broadening of peaks for the polysaccharide which is associated with the dynamics of hydrogen bonding between the free protons and the hydroxyl groups of the polysaccharides. Prior to purification of the crude material sulfated via DMF-HClSO₃, 1H-NMR revealed trace amounts of DMF and sodium acetate and ethanol. Similarly, analysis of the crude chitin and starch sulfates revealed pyridine as the primary impurity. After purification, these peaks were no longer distinguishable in the proton spectra. Information regarding the polymer substitution could not be deconvoluted from the transients taken in the proton NMR. To accomplish this, we analyzed the position and nuclear spin of C₁₃.

Carbon NMR (¹³C-NMR) showed sulfate substitution at C6 and C2 for all partially sulfated celluloses with primary C6S predominantly substituted depicted in (FIG. 4). SS ¹³C-NMR spectra showed nuclear equivalent C6 and C2 sulfate substitution. For ChS, the spectra demonstrated for both DOS primary sulfate substitution predominated in both materials.

### 3.2 Cell Growth and Viability in the Presence of the GAG Mimetics

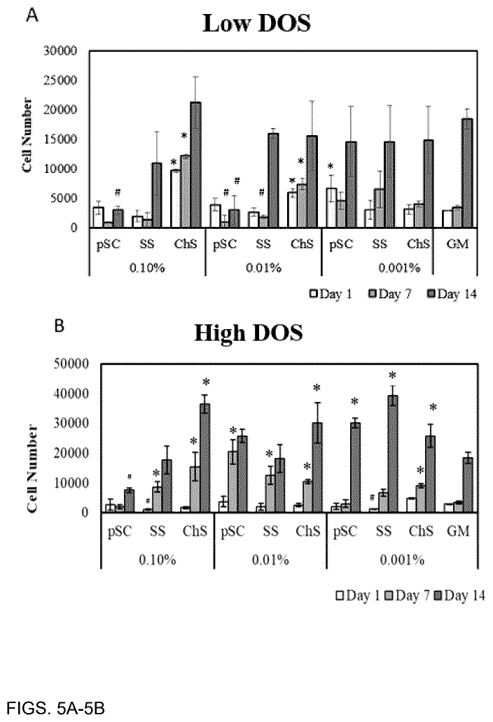

Cell growth and viability of MSCs were evaluated in growth media containing 0.1, 0.01 and 0.001% (wt/v) of the GAG mimetics (FIG. 5). Cell number was significantly higher for ChS at 0.1 and 0.01% at DOS 0.9 compared to all other low DOS groups and the GM control at days 1 and 7 (FIG. 5.a.) (p<0.05). Between day 7 and 14, all groups except pSC at 0.1% and 0.01% at low DOS showed a significant increase in cell number (p<0.05). There was no significant difference between control or experimental groups at day 14 for the low DOS groups.

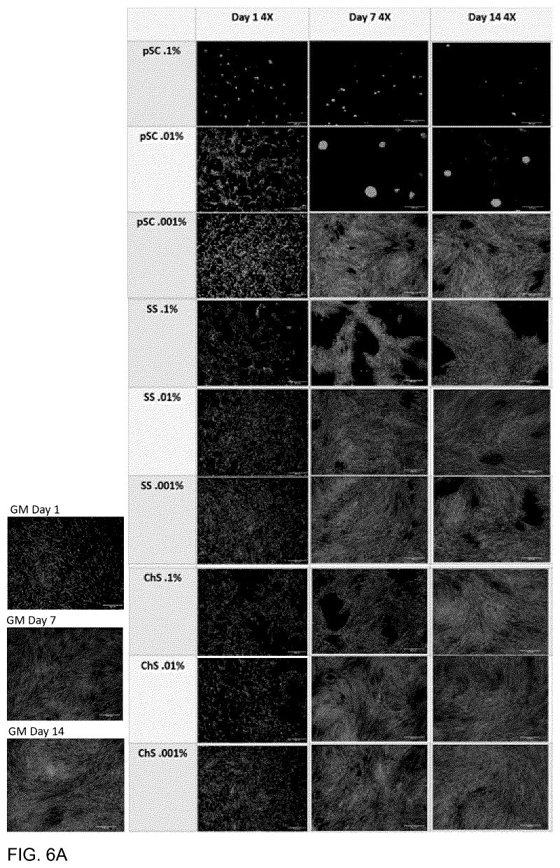

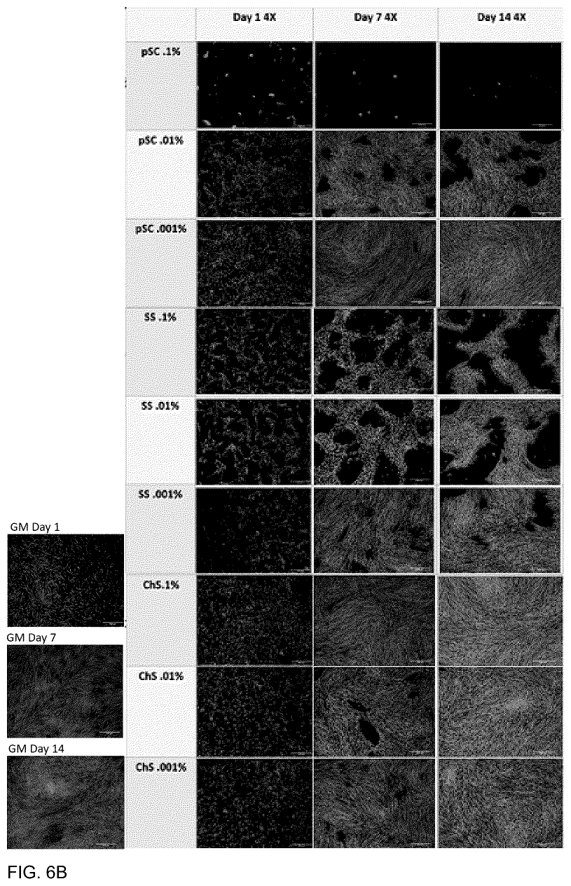

At the high DOS FIG. 5.b., cell number at day 1 for pSC and ChS at higher concentrations were not significantly different from the control GM. At day 7, SS and ChS containing groups at 0.1 and 0.01% had higher cell numbers compared to the control GM. At day 7, pSC at 0.01% showed significantly higher cell numbers compared to the GM control. By day 14, all concentrations of ChS containing groups had significantly higher cell numbers than the GM group at that same time point. Live/dead imaging showed less cells at all time points for cultures treated with pSC at 0.1% and at later time points for pSC at 0.01% at the low DOS concentration (FIG. 6). SS at 0.1% at low DOS and 0.1 and 0.01% at both low and high DOS appeared to have less cells in culture as compared to GM control. All groups and time points had viable cells with few cells detected as dead.

### 3.3 Chondrogenic Differentiation in Pellet Cultures Containing GAG Mimetics

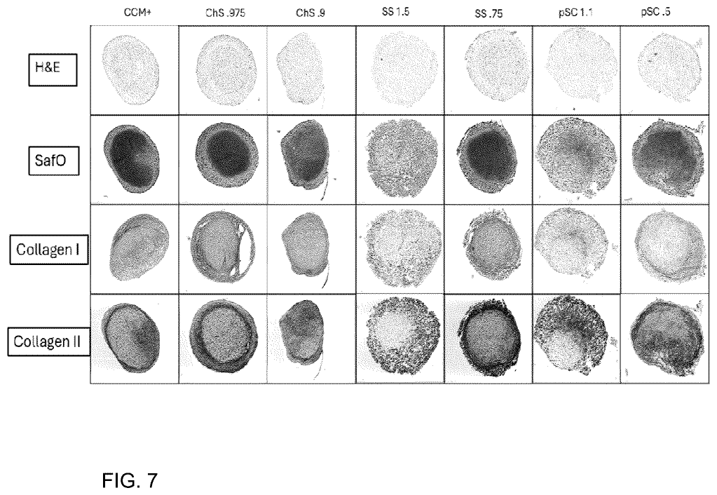

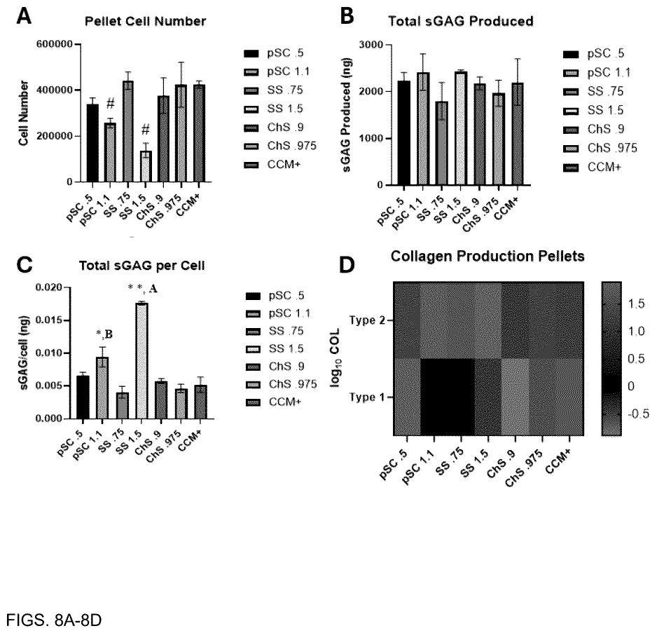

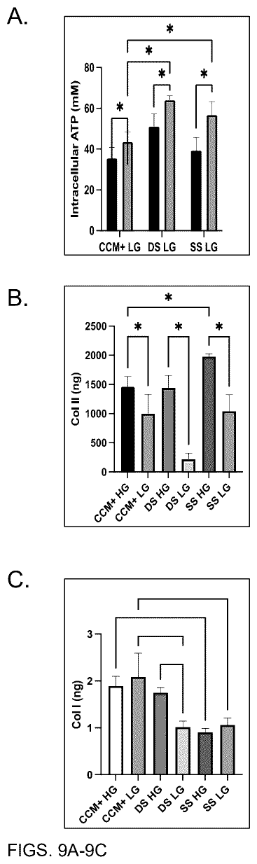

Chondrogenic differentiation was performed in pellet cultures containing media with 0.01% GAG mimetics (FIG. 7). All pellets had an intense proteoglycan stain in their core except for pSC and SS groups at the high DOS. Collagen type II staining was also intense for pellets exposed to the GAG mimetics with less collagen type I staining. The cell number, GAG production and relative measure of collagens type I and II in the pellets were also performed (FIG. 8). Cell number was lower for the higher DOS for pSC and SS groups as compared to the control (CCM+) (FIG. 8.a.). Total GAG produced was similar across all groups (FIG. 8.b.) but when normalized to cell number, the GAG per cell was greatest for the higher DOS for pSC and SS groups, where the SS group was significantly higher than all groups (FIG. 8.c.). Increasing the DOS resulted in higher collagen type II production for all GAG-mimetic groups as compared to control pellets (FIG. 8.d.). The SS at low DOS group also had higher collagen type II production than the control pellet. Collagen type I was an order of magnitude lower than collagen type II also increasing with increasing DOS, except for the SS group where the collagen type I reduced at the higher DOS as compared to the low DOS.

### Discussion

This study aimed to generate sulfated GAG mimetic variants, each of which captured a specific structural feature of native GAGs. These changes in structural features could be assessed for their impact on MSC chondrogenesis. The sulfation of cellulose, starch, and chitin was achieved. Sulfated chitin and starch were synthesized using PSO₃ as the primary sulfating agent. Cellulose was sulfated using the intermediate of DMF and HClSO₃. Chitin and starch sulfations were undertaken at elevated temperatures to catalyze the substitution of the sulfate groups onto the polymer backbone. Attempts were made to increase the DOS without heating by increasing the concentration of PSO₃ however this caused rapid degradation. Heating was used to catalyze the reaction instead of increasing the concentration of the sulfating agent. Varying the DOS was achieved for the sulfated cellulose and starch materials, but limited variation could be achieved for the sulfated chitin. Cell viability and growth was enhanced when supplementing the cell media with low concentrations of higher sulfated pSC, SS and ChS. Chondrogenesis in pellet cultures was most promising for the sulfated cellulose and starch groups, suggesting that both glycosidic linkages may be important for supporting chondrogenesis in TGF-β3 containing media.

Sulfation of polysaccharides has been demonstrated as a viable method to generate mimetic variants for native GAGs. Sulfation can enhance the water solubility and change the chain conformation, which can have an effect on the biological activity of GAG mimetics [31].-Chemical sulfation of polysaccharides have been shown using various sulfating agents [32, 33]. It is well documented that direct sulfation favors the Markovnikov product where sulfation at C6 is favored [34]. The secondary hydroxyls can also be sulfated, however the details on the rates of reaction between the primary and secondary has been unclear [34]. Regio-chemistry in the form of protecting groups has been utilized in small molecules to generate mimetics with varying sulfation and side group patterns that more closely resemble native GAGs. However, these techniques are not viable for polysaccharide modification due to significant challenges including the use of apolar solvents, difficulty of regiochemical control at selected positions in each position of the polymer chain, presence of branching and block structures, and the sensitivity of the glycosidic linkages to acid-based hydrolysis or oxidation which limits the use of potential solvent systems [31, 35, 36].

Chitin and starch sulfates synthesized via PSO₃ yielded similar DOS to native GAGs although the reaction conditions were in more basic conditions than direct sulfation using pure HClSO₃ as demonstrated in prior investigations [24]. Although the compounds were synthesized with varying DOS, ¹³C-NMR suggests for all beta-linked polysaccharides, reaction conditions favored the formation of the Markovnikov product with the majority of sulfate substitution at C6 similar to previous research [26]. SS 0.75 and 1.5 compounds however, revealed similar integration for C2S and C6S which we speculate can be potentially related to a lower relative abundance of C6 because of alpha-1,6 branching. Chitin sulfations did not yield a DOS close to 0.5 sulfates per monomer, which we believe is due to the N-acetyl group which makes the polymer suspension more basic during the synthesis. In order to reduce the pH such that the reaction could occur, more sulfating agent was required which could have altered the reaction kinetics.

Although cellulose, starch and chitin have previously been sulfated, this is the first study where these polysaccharides have been sulfated with different DOS and compared for their effect on cell viability, growth and chondrogenesis. Native GAGs such as heparin, CSC, CSA and HS have been investigated for their effect on cell growth and chondrogenesis in pellet or micro mass cultures as well as in combination with scaffolds [9] [7, 16]. Studies have demonstrated that low concentrations of heparin, which has a higher DOS than CSC, CSA and HS, can enhance cell growth and viability whereas high doses can greatly inhibit cell growth [37]. Similarly, using highly sulfated GAG mimetics, pentosan polysulfate, cell proliferation and chondrogenesis was supported at relatively low concentrations [12]. Whereas, for native GAGs with lower DOS, cell viability and the chondrocyte phenotype can be supported at relatively low concentrations, in the case of CSC [38], as well as high concentrations, in the case of HS in the presence of TGF-β for MSC chondrogenesis [39]. However, in this study, in growth media conditions, cell growth, as indicated by cell numbers over time, was supported for the GAG mimetics SS and ChS at both low and high concentrations and DOS whereas, it was less favorable for pSC at higher concentrations for both DOS. In the presence of TGF-β3, cell numbers were affected by DOS for the pSC and SS groups where increasing DOS reduced in cell numbers. pSC had the highest molecular weight of all of the GAG mimetics and has been shown to sequester TGF-β3 [16]. Interestingly, pSC and SS at high DOS were most favorable for chondrogenesis as indicated by the chondrogenic markers of GAG per cell and production of collagen type II with lower levels of collagen type I. The SS treated pellets produced higher levels of collagen type II and produced less collagen type I than the pSC treated samples at the higher DOS, suggesting it may be more favorable for promoting chondrogenesis. Thus, both the alpha and beta-glycosidic linkage geometry present in sulfated starch and cellulose, respectively, appear beneficial for the deposition of chondrogenic ECM.

Histologically, the pellets with pSC and SS at high DOS appear to be less organized with the appearance of less proteoglycan staining than their low DOS counterparts, ChS and control group. The weaker staining and less organized matrix may have resulted due to the lower cell number in the pSC and SS groups at high DOS, even though the matrix deposition measured was high. This is in contrast to our previous work where 0.01% (w/v) of fully sulfated cellulose (NaCS), with a DOS greater than 2, in chondrogenic induction media containing TGF-β3 resulted in a dense pellet with an intense proteoglycan stain [40]. Interestingly, the NaCS in the previous studies and the low DOS pSC in this study have similar high molecular weights, suggesting molecular weight for the sulfated cellulose may also play a role in chondrogenesis. Also, previous investigations demonstrate that increasing the degree of sulfation of either native GAGs or mimetic variants is associated with increased chondrogenic gene expression and ECM production [11, 16, 41, 42]. One limitation of this study is that Safranin O staining and the sulfated GAG assay do not differentiate between the presence of the proteoglycan and the GAG mimetics used. Aggrecan immunostaining and other quantitative methods may be needed. There may be an optimal level of DOS and concentration of GAG-mimetics in media that will be more favorable for promoting both cell growth and differentiation. The low DOS SS group also demonstrated favorable growth and collagen type II deposition as compared to control (without GAG mimetics). To a lesser extent, the sulfated chitin, which has the amide group, affected differentiation as compared to the control group (without GAG mimetic) and the sulfated cellulose group, which also has beta-glycosidic linkages. However, an evaluation of a wider range of DOS for sulfated chitin is needed.

### Conclusion

GAG mimetics are attractive materials for cartilage tissue engineering due to similarities to native GAGs absent the pitfalls of natural GAGs isolated from animal sources. This study sulfated polysaccharides with distinct structural features and evaluated their effect on chondrogenesis. Our findings demonstrated that that both alpha and beta glycosidic linkages and a higher DOS influenced chondrogenesis/chondrogenic matrix deposition.

### Supporting Information

¹H-NMR spectroscopy showed the decrease in trace impurities for starch sulfate synthesized using PSO₃ and cellulose sulfate, synthesized using DMF-HClSO₃.

### REFERENCES FOR EXAMPLE I

[1] J. P. K. Armstrong, R. Shakur, J. P. Horne, S. C. Dickinson, C. T. Armstrong, K. Lau, J. Kadiwala, R. Lowe, A. Seddon, S. Mann, J. L. R. Anderson, A. W. Perriman, A. P. Hollander, Artificial membrane-binding proteins stimulate oxygenation of stem cells during engineering of large cartilage tissue, Nature Communications 6(1) (2015) 7405.
[2] M. Höök, L. Kjellen, S. Johansson, J. Robinson, CELL-SURFACE GLYCOSAMINOGLYCANS, Annual Review of Biochemistry 53(1) (1984) 847-869.
[3] J. M. Berg, Biochemistry, 7th ed. ed., W. H. Freeman, New York, 2012.
[4] T. K. Toby, C. D. Sommers, D. A. Keire, Detection of native chondroitin sulfate impurities in heparin sodium with a colorimetric micro-plate based assay, Analytical Methods 4(6) (2012) 1488-1491.
[5] M. R. Portocarrero Huang Gloria, Vincent Richard, Hammond Willis, Rizio Louis, Collins George, and Arinzeh Treena Livingston, Gelatin Scaffolds Containing Partially Sulfated Cellulose Promote Mesenchymal Stem Cell Chondrogenesis, Tissue Engineering 23(15-16) (2017) 1011-1021.
[6] T. Wang, F. Yang, A comparative study of chondroitin sulfate and heparan sulfate for directing three-dimensional chondrogenesis of mesenchymal stem cells, Stem Cell Research and Therapy 8(1) (2017).
[7] B. E. Uygun, S. E. Stojsih, H. W. T. Matthew, Effects of Immobilized Glycosaminoglycans on the Proliferation and Differentiation of Mesenchymal Stem Cells, Tissue Engineering. Part A 15(11) (2009) 3499-3512.
[8] S. Chen, P. Fu, R. Cong, H. Wu, M. Pei, Strategies to minimize hypertrophy in cartilage engineering and regeneration, Genes Dis 2(1) (2015) 76-95.
[9] W. Tianyi, Y. Fan, A comparative study of chondroitin sulfate and heparan sulfate for directing three-dimensional chondrogenesis of mesenchymal stem cells, Stem Cell Research & Therapy 8(1) (2017) 1-12.
[10] W.-C. Chen, C.-L. Yao, I. M. Chu, Y.-H. Wei, Compare the effects of chondrogenesis by culture of human mesenchymal stem cells with various type of the chondroitin sulfate C, Journal of Bioscience and Bioengineering 111(2) (2011) 226-231.
[11] D. Kawamura, T. Funakoshi, S. Mizumoto, K. Sugahara, N. Iwasaki, Sulfation patterns of exogenous chondroitin sulfate affect chondrogenic differentiation of ATDC5 cells, Journal of Orthopaedic Science 19(6) (2014) 1028-1035.
[12] P. Ghosh, J. Wu, S. Shimmon, A. C. Zannettino, S. Gronthos, S. Itescu, Pentosan polysulfate promotes proliferation and chondrogenic differentiation of adult human bone marrow-derived mesenchymal precursor cells, Arthritis Res Ther 12(1) (2010) R28.
[13] P. Karunanithi, M. R. Murali, S. Samuel, H. R. B. Raghavendran, A. A. Abbas, T. Kamarul, Three dimensional alginate-fucoidan composite hydrogel augments the chondrogenic differentiation of mesenchymal stromal cells, Carbohydrate Polymers 147 (2016) 294-303.
[14] G. P. Huang, A. Molina, N. Tran, G. Collins, T. L. Arinzeh, Investigating cellulose derived glycosaminoglycan mimetic scaffolds for cartilage tissue engineering applications, Journal of Tissue Engineering and Regenerative Medicine (2017).
[15] R. Menezes, T. L. Arinzeh, Comparative Study of Electrospun Scaffolds Containing Native GAGs and a GAG Mimetic for Human Mesenchymal Stem Cell Chondrogenesis, Annals of Biomedical Engineering 48(7) (2020) 2040-2052.
[16] R. Menezes, L. Sherman, P. Rameshwar, T. L. Arinzeh, Scaffolds containing GAG-mimetic cellulose sulfate promote TGF-β interaction and MSC Chondrogenesis over native GAGs, Journal of Biomedical Materials Research Part A 111(8) (2023) 1135-1150.
[17] J. Wu, S. Shimmon, S. Paton, C. Daly, T. Goldschlager, S. Gronthos, A. C. W. Zannettino, P. Ghosh, Pentosan polysulfate binds to STRO-1+ mesenchymal progenitor cells, is internalized, and modifies gene expression: a novel approach of pre-programing stem cells for therapeutic application requiring their chondrogenesis, Stem Cell Research & Therapy 8(1) (2017) 278.
[18] R. Mhanna, A. Kashyap, G. Palazzolo, Q. Vallmajo-Martin, J. Becher, S. Möller, M. Schnabelrauch, M. Zenobi-Wong, Chondrocyte Culture in Three Dimensional Alginate Sulfate Hydrogels Promotes Proliferation While Maintaining Expression of Chondrogenic Markers, Tissue Engineering Part A 20(9-10) (2014) 1454-1464.
[19] J. Buckwalter, H. Mankin, Articular cartilage. Part I: Tissue design and chondrocyte-matrix interactions, in: J. A. Buckwalter (Ed.) 1997, pp. 600-611.
[20] H. J. Mankin, L. Lippiello, The glycosaminoglycans of normal and arthritic cartilage, J Clin Invest 50(8) (1971) 1712-1719.
[21] Glycoconjugates: composition, structure, and function, Dekker, New York, 1992.
[22] Z. Shriver, I. Capila, G. Venkataraman, R. Sasisekharan, Heparin and heparan sulfate: analyzing structure and microheterogeneity, Handb Exp Pharmacol (207) (2012) 159-76.
[23] ChemAxon, MarvinSketch. Available online: world wide web at chemaxon.com/marvin.
[24] I. B. Cushing, R. V. Davis, E. J. Kratovil, D. W. MacCorquodale, The Sulfation of Chitin in Chlorosulfonic Acid and Dichloroethane, Journal of the American Chemical Society 76(18) (1954) 4590-4591.
[25] E. Bedini, A. Laezza, M. Parrilli, A. ladonisi, A review of chemical methods for the selective sulfation and desulfation of polysaccharides, Carbohydrate Polymers 174 (Supplement C) (2017) 1224-1239.
[26] R. Menezes, S. Hashemi, R. Vincent, G. Collins, J. Meyer, M. Foston, T. L. Arinzeh, Investigation of glycosaminoglycan mimetic scaffolds for neurite growth, Acta Biomaterialia 90(2019) 169-178.
[27] N. Mainreck, S. Brezillon, G. D. Sockalingum, F.-X. Maquart, M. Manfait, Y. Wegrowski, Rapid Characterization of Glycosaminoglycans Using a Combined Approach by Infrared and Raman Microspectroscopies, Journal of Pharmaceutical Sciences 100(2) (2011) 441-450.
[28] B. O. Enobakhare, D. L. Bader, D. A. Lee, Quantification of Sulfated Glycosaminoglycans in Chondrocyte/Alginate Cultures, by Use of 1,9-Dimethylmethylene Blue, Analytical Biochemistry 243(1) (1996) 189-191.
[29] M. M. Green, G. Blankenhorn, H. Hart, Which starch fraction is water-soluble, amylose or amylopectin?, Journal of Chemical Education 52(11) (1975) 729.
[30] E. Wiercigroch, E. Szafraniec, K. Czamara, M. Z. Pacia, K. Majzner, K. Kochan, A. Kaczor, M. Baranska, K. Malek, Raman and infrared spectroscopy of carbohydrates: A review, Spectrochimica Acta Part A: Molecular and Biomolecular Spectroscopy 185(2017) 317-335.
[31] J. Wang, S. Niu, B. Zhao, X. Wang, J. Yao, J. Zhang, W. Zhao, Y. Zhao, Regioselective synthesis of sulfated guar gum: Comparative studies of structure and antioxidant activities, International Journal of Biological Macromolecules 62 (Supplement C) (2013) 734-740.
[32] Z. M. Wang, L. Li, B. S. Zheng, N. Normakhamatov, S. Y. Guo, Preparation and anticoagulation activity of sodium cellulose sulfate, International Journal of Biological Macromolecules 41(4) (2007) 376-382.
[33] R. A. Al-Horani, U. R. Desai, Chemical Sulfation of Small Molecules-Advances and Challenges, Tetrahedron 66(16) (2010) 2907-2918.
[34] W. Wagenknecht, I. Nehls, B. Philipp, Studies on the regioselectivity of cellulose sulfation in an N2O4-N, N-dimethylformamide-cellulose system, Carbohydrate Research 240(1993) 245-252.
[35] A. Y. Desoky, J. Hendel, L. Ingram, S. D. Taylor, Preparation of trifluoroethyl- and phenyl-protected sulfates using sulfuryl imidazolium salts, Tetrahedron 67(6) (2011) 1281-1287.
[36] K. Fukuhara, N. Shimada, T. Nishino, E. Kaji, K. Makino, Regioselective, Tin-Free Sulfation of Unprotected Hexopyranosides by Using Phenylboronic Acid, European Journal of Organic Chemistry 2016(5) (2016) 902-905.
[37] L. Ling, E. T. Camilleri, T. Helledie, R. M. Samsonraj, D. M. Titmarsh, R. J. Chua, O. Dreesen, C. Dombrowski, D. A. Rider, M. Galindo, I. Lee, W. Hong, J. H. Hui, V. Nurcombe, A. J. van Wijnen, S. M. Cool, Effect of heparin on the biological properties and molecular signature of human mesenchymal stem cells, Gene 576(1, Part 2) (2016) 292-303.
[38] S.-R. Yang, S. Peng, C.-Y. Ko, I. M. Chu, The effects of different molecular weight chondroitin-4-sulfates in chondrocyte pellet culture, Cytotechnology 68(3) (2016) 371-379.
[39] J. Chen, Y. Wang, C. Chen, C. Lian, T. Zhou, B. Gao, Z. Wu, C. Xu, Exogenous Heparan Sulfate Enhances the TGF-β3-Induced Chondrogenesis in Human Mesenchymal Stem Cells by Activating TGF-β/Smad Signaling, Stem Cells Int 2016(2016) 1520136-1520136.
[40] G. P. Huang, A. Molina, N. Tran, G. Collins, T. L. Arinzeh, Investigating Cellulose Derived Glycosaminoglycan Mimetic Scaffolds for Cartilage Tissue Engineering Applications, Journal of Tissue Engineering and Regenerative Medicine 12(1) (2018) e592-e603.
[41] C. Merceron, S. Portron, C. Vignes-Colombeix, E. Rederstorff, M. Masson, J. Lesoeur, S. Sourice, C. Sinquin, S. Colliec-Jouault, P. Weiss, C. Vinatier, J. Guicheux, Pharmacological modulation of human mesenchymal stem cell chondrogenesis by a chemically oversulfated polysaccharide of marine origin: potential application to cartilage regenerative medicine, Stem cells (Dayton, Ohio) 30(3) (2012) 471-480.
[42] S. Zhao, Z. Wang, J. Chen, J. Chen, Preparation of heparan sulfate-like polysaccharide and application in stem cell chondrogenic differentiation, Carbohydrate Research 401(2015) 32-38.

### Examples—II

Osteoarthritis (OA) is a degenerative joint disease that results in the loss of articular cartilage and normal joint function. After damage, articular cartilage has a limited capacity to heal. Current therapies only alleviate symptomatic pain, and surgical interventions do not restore normal hyaline cartilage. Tissue engineering and the use of biomaterial scaffolds may facilitate the growth of cells and production of functional, hyaline cartilage. Glycosaminoglycans (GAGs) are polysaccharides found in the native extracellular matrix and during early cartilage development. GAGs play a mechanical role in the native ECM and can also facilitate the growth of cartilage as they sequester growth factors and may affect cellular behaviors including growth, migration and differentiation [1,2]. In this study, GAG mimetics were derived from starch and dextran. Starch-based materials have been investigated for several biomedical applications and are biocompatible. Dextran sulfate is generally recognized as safe (GRAS) by the FDA and dextran products have been used clinically in cosmetic applications, blood clotting factors, and pharmaceutical applications. In addition, cells may uptake starch and dextran and enzymatically degrade these materials where their byproducts may be a source of glucose. In this study, we explored the metabolic activity of mesenchymal stem cells (MSCs) undergoing chondrogenesis in low and high glucose conditions in the presence of these GAG-mimetics. Then, developed injectable fibers consisting of the GAG-mimetic, starch sulfate (SS), and investigated MSC chondrogenesis on varying fiber densities to determine a suitable design for cartilage repair.

Methods: The GAG-mimetic starch sulfate (SS) was synthesized by suspending starch (Sigma Aldrich) in pyridine and refluxed over pyridine-sulfurtrioxide. Products were then neutralized and purified. Dextran sulfate (DS) was obtained from Dextran Products, Inc. Human mesenchymal stem cells (MSCs) were evaluated in pellet cultures in media consisting of 0.01 w/v % of GAG mimetic (SS or DS) added to chondrogenic induction media (CCM+), comprised of high glucose (HG-4.5 g/L) DMEM, sodium pyruvate, proline, glutamine, antibiotic-antimycotic, ITS-Premix, and 10 ng/ml transforming growth factor beta3 (TGF-β3) and evaluated up to day 28. Cultures were also evaluated in chondrogenic induction using low glucose (LG-1.5 g/L) DMEM. Pellets cultured in CCM+ with no GAG mimetics served as a control. Fibers consisting of gelatin containing GAG mimetics were created by electrospinning, crosslinked using EDC-NHS and then fragmented using a processing blender. The fibers were dispersed in PBS, ejected into a 96-well plate at 1.5 (Low), 2.5 (Medium), and 3.5 (High) mg/mL densities. The fibers seeded with human mesenchymal stem cells (MSCs) in chondrogenic induction media (CCM+) and evaluated for up to day 28. Cell number was evaluated via the Quant-iT dsDNA Pico Green assay kit (Invitrogen) and total GAG content using the DMMB assay (N=4 per group per time point). The production of collagen types I and II was quantified using enzyme linked immunosorbent assays (N=4 per group) (ELISA, Chondrex, USA). Immunostaining for types I and II collagen and imaging by confocal microscopy were also performed. Statistical analysis was performed using SPSS statistical package (SPSS Inc., Chicago, IL, USA). One-way or two-way analysis of variance (ANOVA) was performed with multiple comparisons between groups using Tukey's posthoc test (p<0.05).

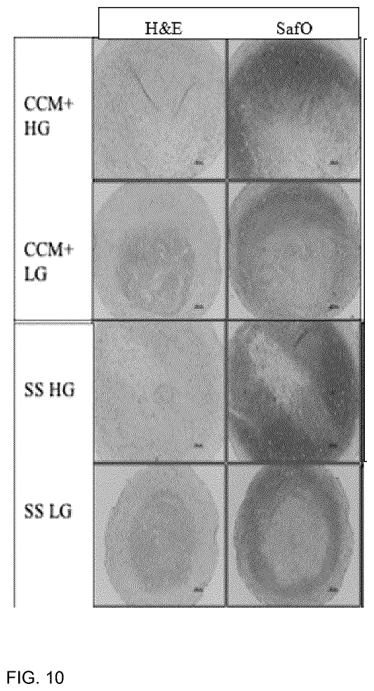

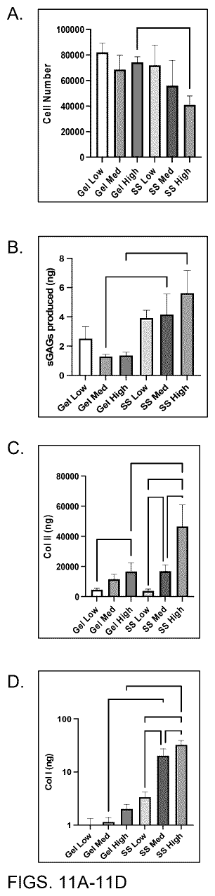

Results: A significantly higher intracellular ATP was detected for cells in SS cultures in LG media as compared to cells without GAG-mimetics (CCM+ only) in LG media (FIG. 9A). Cells produced significantly higher amounts of collagen type II for the SS group as compared to CCM+ (FIG. 9B). Histology also showed that the SS group in HG media appeared to have more proteoglycan, as shown by the SafO stain (FIG. 10). Fragmented fibers had uniform fiber morphology. Cell number was maintained throughout 28 days on all fiber densities, and cells produced GAG over time, where cells on Medium density fibers had the highest GAG production (p<0.05). Despite SS containing scaffolds exhibiting a lower cell number, there was considerably more sGAG content produced over the 28 day study (FIG. 11A,B). In the presence of LOW density fibers, cells appeared to contract around the fibers, showing visible condensation of fiber bundles over 28 days. By 28 days in culture, all groups produced significantly more collagen type II than type I (FIG. 11C,D) with High densities showing significantly more production of collagen type II than the Medium and Low density groups. SS containing fibers exhibited significantly more collagen type II than the gelatin fibers alone at all fiber densities.

Discussion: Collagen type II expression was directly proportional to fiber density. High density fibers produced a significant increase in Col II production than medium and low densities, indicating fiber density may impact chondrogenesis. Starch sulfate supplemented cell pellets exhibited increased amounts of intracellular ATP, and greater Col II production was observed in both pellet supplementation and incorporated fragmented gelatin fibers, demonstrating that SS may promote chondrogenesis. The combination of high density fibers and SS containing scaffolds led to even higher Col II production, which further exemplified the chondrogenic properties of either augmentation method. The high expression of Col II with low expression of Col I suggests the fibers may support a more homogeneous hyaline cartilage matrix formation.

Significance: Tissue regeneration approaches that can rebuild lost hyaline cartilage are needed. ECM mimetics can be used to enhance repair and joint function. The results of this study demonstrate the using fibers as an injectable substrate for cartilage regeneration.

### REFERENCES FOR EXAMPLE II

(1) Menezes R, Tissue Eng, 2017, 23; 1011-1021.
(2) Huang G P, JTERM, 2018, 12; e592-e60.
(3) Valcourt J R. Cell Cycle. 2012; 11 (9): 1680-1696.

### Examples—III

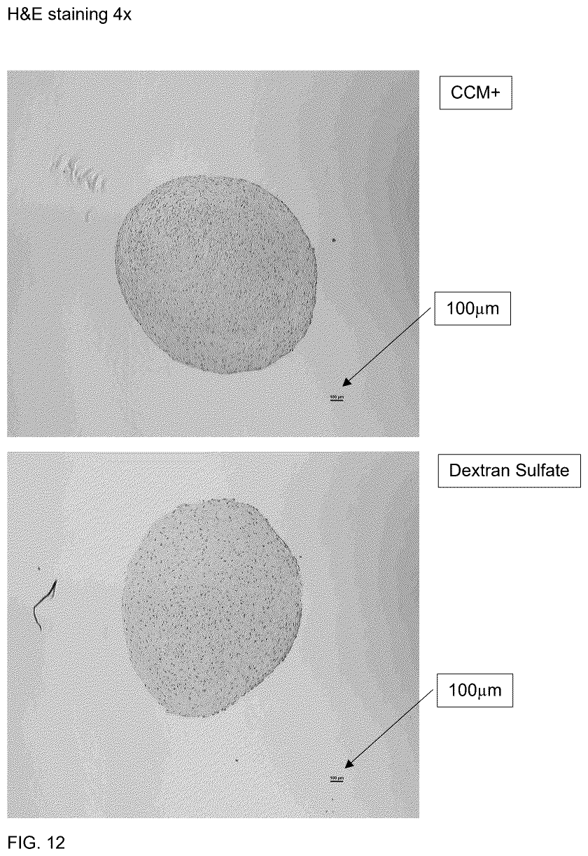

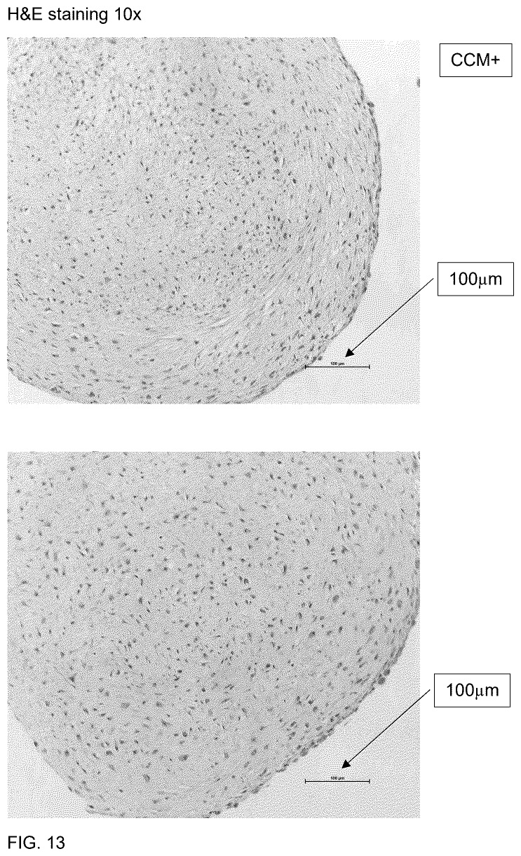

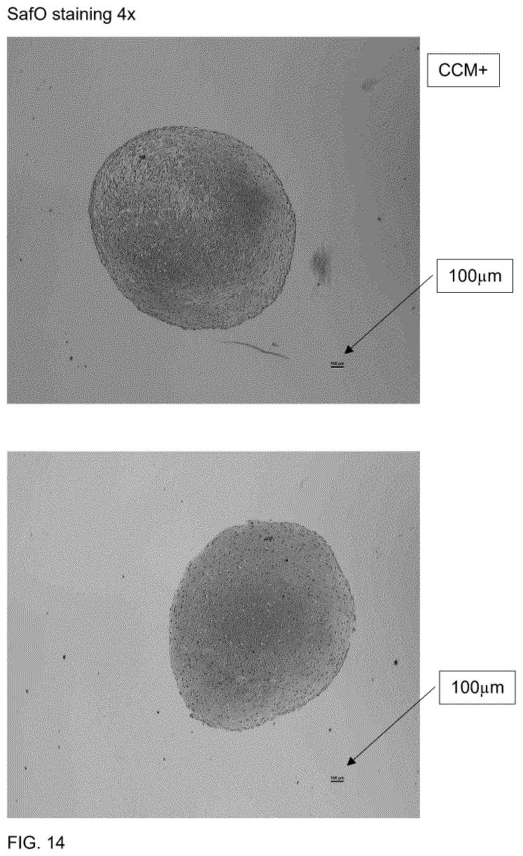

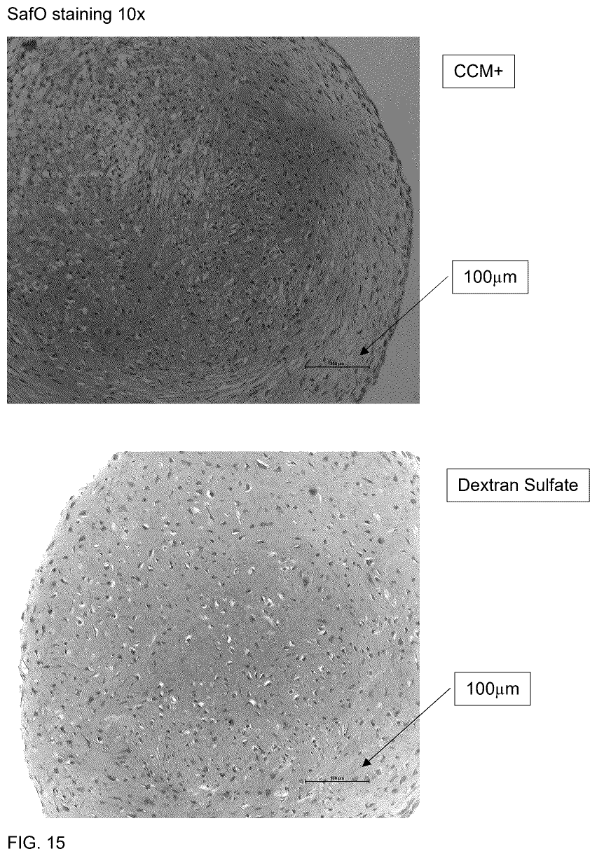

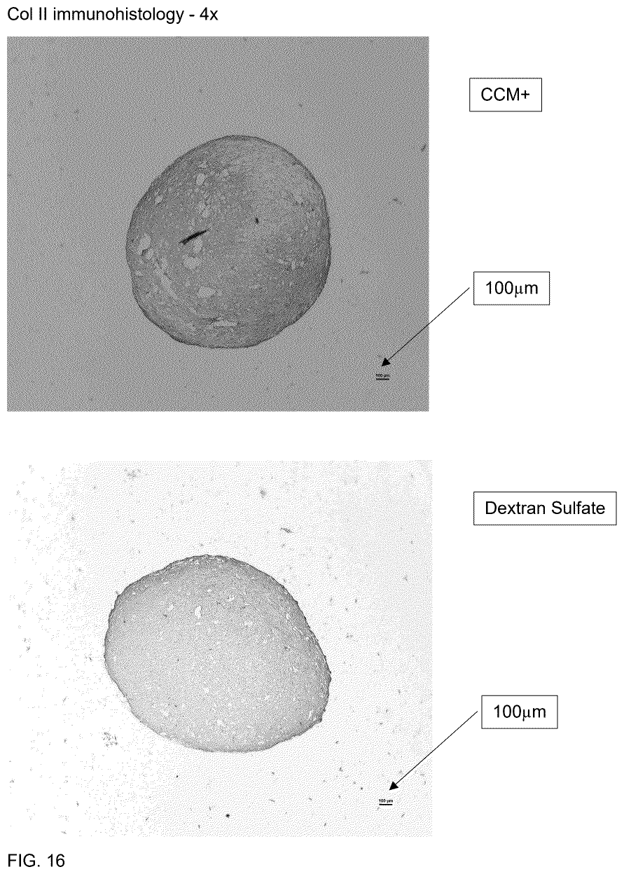

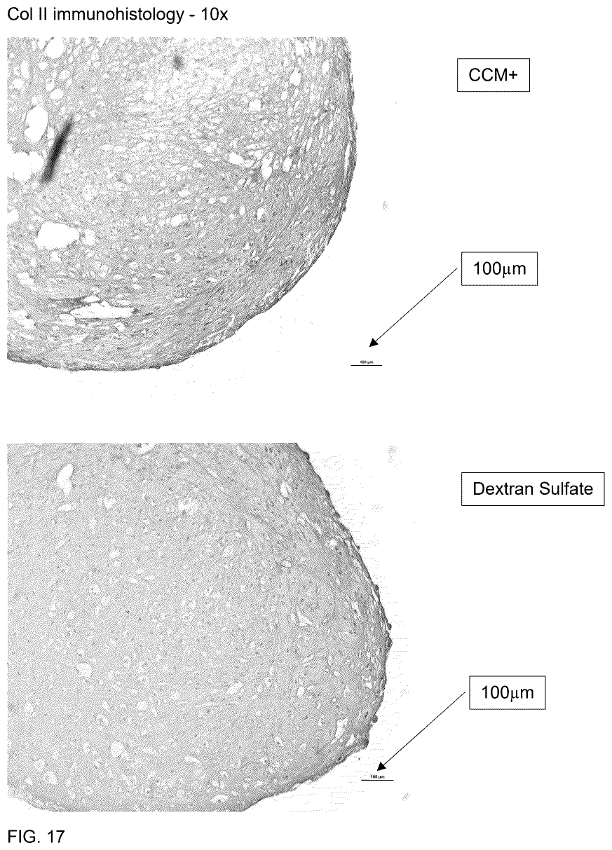

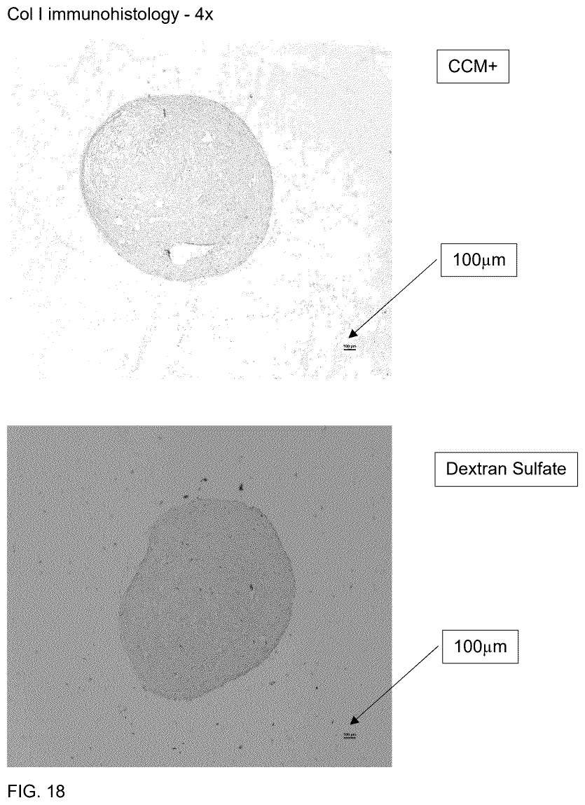

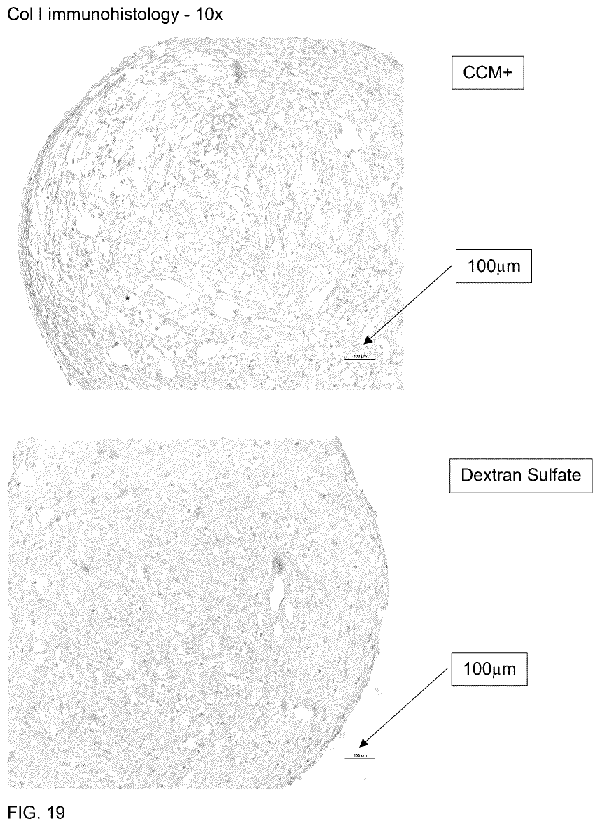

Dextran sulfate as intra-articular injectable biomaterial: We examined human MSC chondrogenesis using 0.01 wt. % Dextran Sulfate (10 kDa) in pellet culture. Passage 3 MSCs from donor 10 was expanded to passage 4. Group set: CCM+ (control), 0.01 wt % Dextran Sulfate. All of the cell pellets were cultured in 500 μl CCM+ until day 7 to ensure strong pellet aggregation. On day 2, 5, two media changes with CCM+ were conducted. On day 7, 500 μl media were removed and CCM+, 0.01 wt % dextran sulfate media were added. Media change: Since day 7, half of the media (250 μl) was removed, and 250 μl fresh CCM+ media was added. Media will be changed twice a week. Media exchange was to mimic synovial fluid clearance. Histology and immunohistochemical staining at 28 days in culture. See FIGS. 12-17 for results.

### Results

Findings demonstrate a more homogenous proteoglycan and collagen type II staining throughout the pellet for cultures containing dextran sulfate as compared to control (CCM+). Minimal collagen type 1 staining for pellet cultures containing dextran sulfate as compared to control cultures where collagen type 1 staining was present throughout the pellet. Findings demonstrate dextran sulfate promotes chondrogenesis and the potential for the dextran sulfate as an injectable agent/carrier for osteoarthritis.

### Examples—IV

Methods for Inserting Scaffolds into Osteochondral Defects

A lateral parapatellar incision and arthrotomy is performed on a rabbit knee. The femoral condyle is exposed by medial dislocation of the patella. A 2.8 mm trephine burr is used to drill a defect, 3 mm in diameter by 3-4 mm in depth, in the femoral condyle, while Ringer's solution is irrigated during drilling. The defect is filled by inserting a gelatin scaffold, cut to 3 mm in diameter using a biopsy punch at the time of surgery and having an approximate thickness of 1 mm. Gelatin scaffold layers are layered to fill the subchondral bone portion of the defect (e.g., 3 gelatin scaffold layers stacked) followed by the scaffold of interest (a starch sulfate gelatin scaffold or cellulose sulfate gelatin scaffold as described herein, or a an unloaded gelatin scaffold as a control), which is 3 mm in diameter×0.5 mm thick since the cartilage thickness in rabbits is approximately, 0.5 mm thick. All scaffolds can be press-fit into defects. The top scaffold layer, which is the test material of interest, interfaces with the host cartilage. The starch sulfate gelatin scaffold or cellulose sulfate gelatin scaffold will show better repair. In weight-bearing positions, inclusion of TFG-beta 3 in addition.

1. A composition for chondrogenesis or chondral repair or osteochondral repair, comprising a sulfated chitin, a sulfated starch, a sulfated cellulose or a sulfated dextran and, optionally, a carrier.

2. The composition of claim 1 comprising a sulfated starch or sulfated cellulose.

3. The composition of claim 1 comprising a sulfated dextran.

4. The composition of claim 1 comprising a sulfated chitin.

5. The composition of claim 3 comprising a 10 KDa dextran.

6. The composition of claim 2, comprising the sulfated starch wherein a degree of sulfation of the sulfated starch is 0.75 to 1.5 per monomer of starch.

7. The composition of claim 6, wherein the degree of sulfation of the starch is 1.5 per monomer of starch.

8. The composition of claim 6, comprising C6 sulfation of the starch.

9. The composition of claim 2, comprising the sulfated cellulose wherein a degree of sulfation of the cellulose is 0.5 to 1.1 per monomer of cellulose.

10. The composition of claim 9, comprising C6 and C2 sulfation of the cellulose.

11. The composition of claim 1, wherein a degree of sulfation of the chitin is 0.9 to 0.975 per monomer of chitin.

12. The composition of claim 11, comprising C6 and C2 sulfation of the chitin.

13. The composition of claim 1, in the form of an injectable fiber or in the form of a scaffold.

14. An injectable fiber composition for chondrogenesis or articular cartilage repair or treating a chondral or osteochondral defect, the injectable fiber composition comprising a sulfated chitin, a sulfated starch, a sulfated cellulose or a sulfated dextran, and a carrier.

15. A scaffold for chondrogenesis or articular repair comprising sulfated starch with a degree of sulfation of 1.5 per monomer of starch.

16. The scaffold of claim 15, wherein the scaffold comprises fibers, optionally fibers composed of gelatin.

17. The scaffold of claim 15, comprising one or more gelatin layers.

18. The injectable fiber composition of claim 14, wherein the fibers further comprise an amount of TGF-beta3.

19. The scaffold of claim 15, wherein the fibers further comprise an amount of TGF-beta3.

20. A method of improving chondrogenesis, or treating osteoarthritis, or treating joint degradation or treating an osteochondral defect in a subject in need thereof, the method comprising administering the composition comprising sulfated chitin, sulfated starch, sulfated cellulose or sulfated dextran of claim 1 to a cartilage containing area, to a joint surface or to an osteochondral defect of the subject.
